(19) United States
(12) Patent Application Publication
(10) Pub. No.: US 2010/0294952 A1
(43) Pub. Date: Nov. 25, 2010

(54) CONTROLLED AGENT RELEASE AND SEQUESTRATION

(75) Inventors: Chad A. Mirkin, Wilmette, IL (US); Matthew R. Jones, LaMesa, CA (US); Jill E. Millstone, Jacksonville, FL (US); David A. Giljohann, Chicago, IL (US); Dwight S. Seferos, Toronto (CA); Kaylie L. Young, Endicott, NY (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: NORTHWESTERN UNIVERSITY, Evanston, IL (US)

(21) Appl. No.: 12/687,595

(22) Filed: Jan. 14, 2010

Related U.S. Application Data
(60) Provisional application No. 61/144,981, filed on Jan. 15, 2009.

(51) Int. Cl. A61N 5/06

(52) U.S. Cl. 250/492.1

(57) ABSTRACT

Disclosed are nanostructures having nanoprisms and agents, such as diagnostic and/or therapeutic agents. Nanoprisms with a surface plasmon resonance in the near-infrared convert irradiation, such as from a laser into heat selectively to allow the dissociation, such as dehybridization of oligonucleotide duplexes, of agents associated with the nanoprism surface. These nanostructures show morphological, chemical, and functional stability under hours of irradiation. Further disclosed are methods of selectively releasing agents from nanostructures after directed surface plasmon resonance mediated heating of the nanoprisms. Released agents, such as oligonucleotides, are unharmed by this process and can be repeatedly released and sequestered under spatiotemporal control.

### CROSS REFERENCE TO RELATED APPLICATIONS

This application claims the benefit of U.S. Provisional Application No. 61/144,981, filed Jan. 15, 2009, the disclosure of which is incorporated by reference in its entirety.

### STATEMENT OF GOVERNMENT INTEREST

This invention was made with U.S. government support under the National Science Foundation (NSEC) Grant No. EEC-0647560, Air Force Office of Scientific Research (AFOSR) Grant No. FA9550-07-1-0534, and the National Institutes of Health Pioneer Award Grant No. 5DP1 OD000285. The government has certain rights in this invention.

### BACKGROUND

The irradiation of anisotropic gold nanostructures at their surface plasmon resonance (SPR) is an efficient method for converting light into heat,(1) particularly in the near-infrared region where absorbance by biological tissues is minimal.(1-7) These techniques however, typically rely on high-power pulsed lasers that can cause irreversible damage to the local environment and have only recently been used to investigate a particle selective response.(4, 5, 8-10) Few reports to date focus on the use of SPR-mediated heating to release drugs, nucleic acids, or other therapeutic agents under mild conditions amenable to temperature sensitive environments.(11, 12)

### SUMMARY

Disclosed herein are methods of selectively releasing or sequestering an agent using nanostructures comprising nanoprisms with an agent associated with the nanoprism. More specifically, disclosed herein is a method comprising irradiating a nanostructure comprising (a) a nanoprism and (b) an agent with light having a narrow band of wavelengths or a single wavelength that excites a surface plasmon resonance of the nanoprism, wherein prior to the irradiating, the agent is associated with the nanoprism, and after the irradiating, the agent is dissociated from the nanoprism. The irradiating can produce a temperature surrounding the nanoprisms of about 40° C. to about 85° C. The SPR of the nanoprism after the irradiating can be substantially identical to the SPR prior to irradiating.

The nanoprism can be any geometry, including, for example, triangular. The nanoprism can comprise gold or silver. In cases where the nanoprism is triangular, the edge length of the nanoprism can be about 90 nm to about 200 nm, or about 100 nm to about 150 nm.

The agent can be a diagnostic agent, a therapeutic agent, or both. For example, the nanostructure can comprise two or more agents. In various embodiments, the agent comprises an oligonucleotide, a protein, a peptide, a non-peptide drug, or mixtures thereof. In some embodiments, the agent is attached to a spacer. The spacer can comprise an organic moiety, and in specific embodiments, the organic moiety comprises a polymer, or in specific cases, a water-soluble polymer. In some cases, the polymer comprises an oligonucleotide. In cases where the nanostructure comprises two or more agents, one agent can comprise an oligonucleotide and another agent can comprise a protein, a peptide, an oligosaccharide, a non-peptide drug, or an antibody.

In embodiments where the agent comprises an oligonucleotide, the oligonucleotide can be complementary to a polynucleotide encoding a gene product. The oligonucleotide can be sufficiently complementary to inhibit expression of the gene product, such as in vivo inhibition or in vitro inhibition. The oligonucleotide can be 100% complementary to the polynucleotide, greater than 95% complementary to the polynucleotide, greater than 90% complementary to the polynucleotide, greater than 80% complementary to the polynucleotide, greater than 75% complementary to the polynucleotide, greater than 70% complementary to the polynucleotide, greater than 65% complementary to the polynucleotide, greater than 60% complementary to the polynucleotide, greater than 55% complementary to the polynucleotide, or greater than 50% complementary to the polynucleotide.

The oligonucleotide can be about 5 to about 100 nucleotides in length, about 5 to about 90 nucleotides in length. about 5 to about 80 nucleotides in length, about 5 to about 70 nucleotides in length, about 5 to about 60 nucleotides in length, about 5 to about 50 nucleotides in length, about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, or about 5 to about 10 nucleotides in length.

In some embodiments, the method disclosed herein is when the agent comprises a first oligonucleotide having a first sequence, the nanostructure comprises a second oligonucleotide having a second sequence and attached to at least a portion of the nanoprism surface; all or a portion of the second sequence is sufficiently complementary to the first sequence to allow hybridization of the first oligonucleotide and the second oligonucleotide; and after the irradiating, the first oligonucleotide dehybridizes from the second oligonucleotide. The oligonucleotide can be DNA, RNA, or mixtures thereof.

In various embodiments, the nanostructure further comprises a fluorophore. The fluorophore can be covalently attached to the agent. Specific examples of fluorophores include fluorescein dye, 6-((7-amino-4-methylcoumarin-3-acetyl)amino)hexanoic acid, 5(and 6)-carboxy-X-rhodamine, a rhodamine dye, a benzophenoxazine, Cyanine 2 (Cy2) dye, Cyanine 3 (Cy3) dye, Cyanine 3.5 (Cy3.5) dye, Cyanine 5 (Cy5) dye, Cyanine 5.5 (Cy5.5) dye, Cyanine 7 (Cy7) dye, Cyanine 9 (Cy9) dye, 6-carboxy-4′,5′-dichloro-2′,7′-dimethoxyfluorescein, and 5(6)-carboxy-tetramethyl rhodamine. In cases where the nanostructure comprises a fluorophore, the method further comprises monitoring a change in fluorescence of the fluorophore and correlating the change in fluorescence to release or sequestration of the agent from or to the nanoprism, wherein an increase in fluorescence corresponds to a release of the agent and a decrease in fluorescence corresponds to a sequestration of the agent.

### BRIEF DESCRIPTION OF THE FIGURES

FIG. 1 shows irradiation of a non-prismatic nanoparticle and a prismatic triangular nanoprism with light at a wavelength that excites the surface plasmon resonance of the prism, generating heat at the nanoprism surface and resulting release of oligonucleotides from the nanoprism, and not from the nanoparticle.

FIG. 2 shows a schematic illustration of the laser coupled fluorescence spectrometer for SPR excitation of oligonucleotide-modified gold nanoprisms and the simultaneous fluorescence detection of dehybridized oligonucleotides.

FIG. 3 shows (A) extinction spectra of oligonucleotide-modified gold nanoprisms (solid) and spherical nanoparticles (dashed). The vertical line illustrates 1064 nm laser for comparison; (B) spectra of fluorescence intensity as a function of time for a solution of oligonucleotide-modified gold nanoprisms (solid) and spherical nanoparticles (dashed) that have both been hybridized to fluorescein labeled complementary oligonucleotides. The arrow denotes the onset of 1064 nm laser irradiation of the solution.

FIG. 4 shows (A) extinction spectra of oligonucleotide-modified gold nanoprisms before (solid) and after (dashed) 9 hours of 1064 nm laser irradiation. The inset shows representative transmission electron microscopy (TEM) images before and after irradiation. Scale bars represent 50 nm; and B) fluorescence intensity of nanoprisms functionalized with a Cy5.5 labeled thiolated oligonucleotide during 1064 nm laser irradiation. The dashed line denotes the fluorescence value after oxidation of the nanoprisms using potassium cyanide.

FIG. 5 shows (A) equilibrium fluorescence intensity of oligonucleotide-modified gold nanoprisms with the irradiation of the 1064 nm laser shown (indicated when laser turned on and off); and (B) non-equilibrium fluorescence intensity of oligonucleotide-modified gold nanoprisms with the cyclic irradiation of the 1064 nm laser shown (indicated when laser turned on and off).

FIG. 6 shows the measurement of the fluorescence intensity of a 45 pM sample of oligonucleotide-modified gold nanoprisms hybridized to fluorophore-labeled complements as a function of temperature.

FIG. 7 shows the measurement of the fluorescence intensity of an oligonucleotide duplex consisting of a fluorophore-quencher pair under 500 mW 1064 nm laser irradiation (solid) and under increased temperatures (dashed).

### DETAILED DESCRIPTION

Disclosed herein are structures (interchangeably referred to as nanostructures, throughout) comprising a nanoprism core associated with an agent, such as a diagnostic agent and/or a therapeutic agent. The association between the agent and the nanoprism core can be through a covalent bond directly or indirectly (e.g., through a spacer or linker moiety) to the nanoprism surface. In some embodiments, the association of the agent to the nanoprism is through a non-covalent interaction, such as through hydrogen-bonding or hydrophobic interactions.

Disclosed herein are methods that excite the SPRs of the nanoprisms that can cause a discrete change in temperature at the nanoprism site. The ability to heat nanostructures and cause a local temperature change using external stimuli is of considerable interest for hyperthermic cancer therapies,(1-3, 7) drug delivery,(5, 13) DNA actuation,(14) and microsecond DNA melting analysis.(9) However, other methods using radio frequency electromagnetic fields to generate heating of iron oxide nanostructures are not particle selective and require the collective interaction of many magnetic nanoparticles in order to generate local heat. In general, these approaches require high local concentrations (typically mg/mL) of particles and are unable to effect heating at a single particle level.(13)

SPR-based heating of nanoparticles irradiated under femtosecond pulsed-laser irradiation have been reported to provide temperature increases in excess of 900° C.(15) Such high local temperature increases can cause acoustic cavitation and have been used to damage sensitive environments, such as lipid membranes, and ablate cancer cells.(1-3, 5, 7) These conditions can even melt the nanoparticle core,(5, 8) and dissociate nanoparticle ligands (e.g. thiols), which results in the destabilization and aggregation of the colloidal particles.(4) SPR heating of metal nanoparticles has been used primarily in destructive, largely irreversible processes. Disclosed in the methods herein is SPR heating of nanostructures that is a non-destructive, reversible process.

The optical properties of nanoprisms and the thermal properties of nucleic acids or other agents can be used to create nanostructures capable of light-controlled agent release. It is further demonstrated herein that modified nanoprisms, such as gold nanoprisms, are chemically, morphologically, and functionally stable under several hours of laser irradiation, and thus represent a class of nanostructures capable of SPR-mediated heating to dehybridize oligonucleotides or release other agents associated with the nanoprism via a heat labile association.

As used herein, a “heat labile association” refers to an interaction between an agent and a nanoprism, or between two agents, such as a covalent bond or a non-covalent interaction, which can be altered by a change in temperature, either a decrease or increase in temperature. For example, two oligonucleotides hybridized into a duplex can dehybridized at increased temperatures, e.g., the duplex “melts.” Other non-covalent interactions, such as hydrophobic interactions, can also be heat labile associations. Covalent bonds that can be broken by an increase or decrease in temperature, or can be formed by an increase or decrease in temperature, are also contemplated.

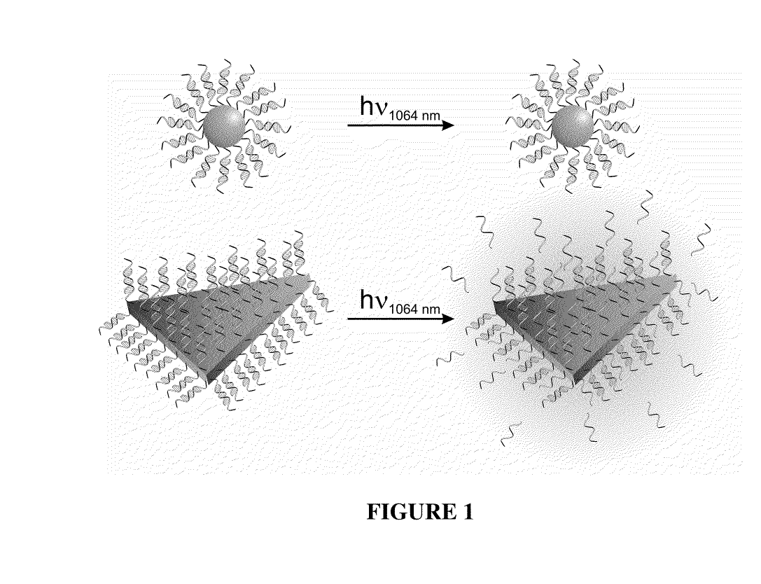

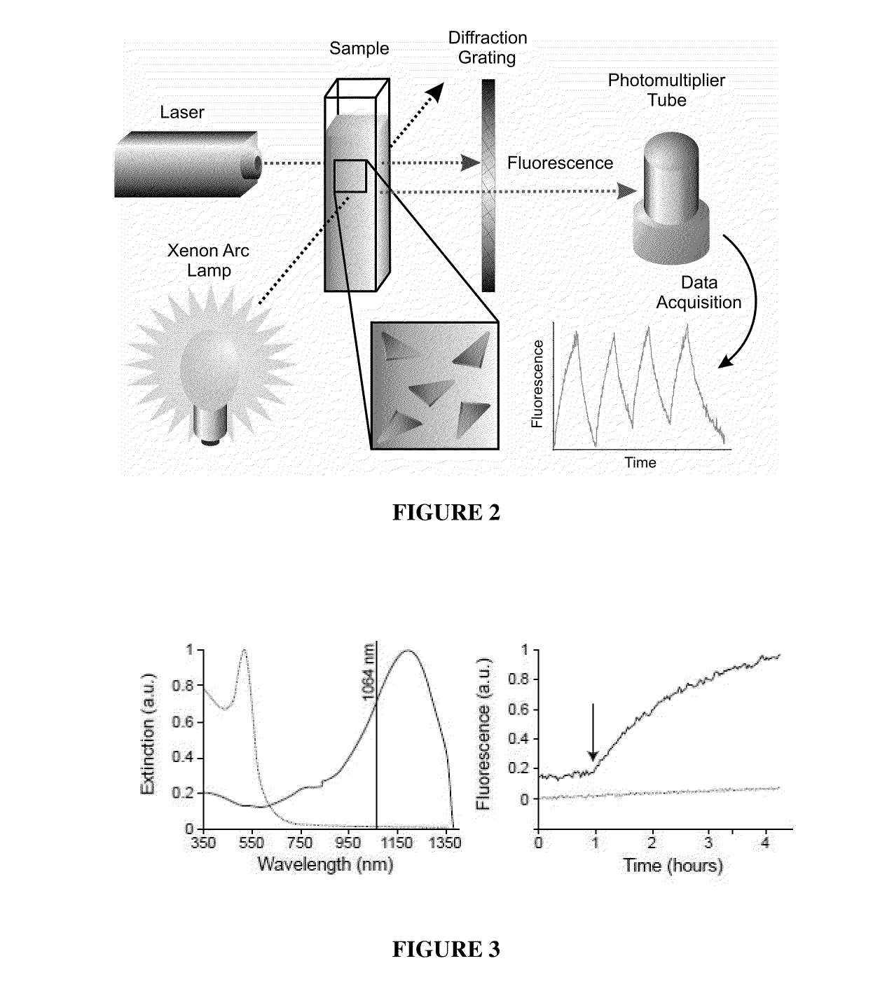

In a typical experiment, triangular gold nanoprisms (120±12 nm edge length, 7.5±0.5 nm thick),(22) which have a strong dipole surface plasmon resonance in the near-infrared region (about 1200 nm),(23) were functionalized with a thiolated oligonucleotide containing a 15-base recognition sequence hybridized to a fluorescein-labeled complementary sequence.(21) Using these particles, the heat generated by SPR-specific irradiation can dissociate the attached duplex, and release the fluorophore-labeled oligonucleotide (FIG. 1). Simultaneous SPR excitation of the nanoprisms and measurement of fluorescence intensity consisting of a near-infrared laser coupled to a florescence spectrometer was performed (FIG. 2). The excitation and emission of the fluorophore-labeled complementary oligonucleotide can be monitored during the irradiation of the sample by a 500 mW continuous wave 1064 nm laser source. Because gold efficiently quenches fluorescence in a distance-dependent fashion,(24, 25) the quenching ability of the nanoprism-oligonucleotide conjugate is used to monitor the dehybridization of the fluorophore-labeled oligonucleotide in real time. Such a method can be used to qualitatively assess the effect of laser irradiation on the hybridization state of the attached duplex.

Using this configuration, the ability of SPR-mediated heating of oligonucleotide-modified nanoprisms to dehybridize the fluorophore-labeled complementary oligonucleotides was tested. Without laser irradiation, the system exhibits a constant fluorescence signal. Under near-infrared laser irradiation (1064 nm), a rapid increase in fluorescence intensity was observed, which was assigned to immediate photothermal generation of heat sufficient to denature the duplex (FIG. 3B). Over the course of 3 hours, the fluorescence intensity becomes constant, indicative of equilibrium at a higher fluorescence intensity. In this regime, only a small area of the cuvette is irradiated (spot size about 0.45 mm) where nanoprisms diffuse into the beam, undergo SPR-mediated local heating and release their oligonucleotide cargo via temperature-induced duplex dehybridization. Simultaneously, oligonucleotide-modified nanoprisms diffuse out of the beam, cool down and rehybridize to fluorophore-labeled complements. Although this process is dependent on the geometry of the experimental setup, on average, about 10% of all fluorophore-labeled oligonucleotides were dehybridized during this process. Interestingly, 1064 nm laser irradiation of a free fluorophore-quencher labeled duplex in the absence of Au nanoprisms results in no change in fluorescence, confirming the necessity of the inorganic nanoprism to convert the incident light into heat (FIG. 7). It is important to note that the concentrations used in these experiments are low (45 pM) and result in nanoprisms separated by an average distance of 4 μm. These values are much greater than the longest observed SPR interactions,(26) and suggest the origin of the local heating does not rely on interparticle interactions but is active at the single particle level.

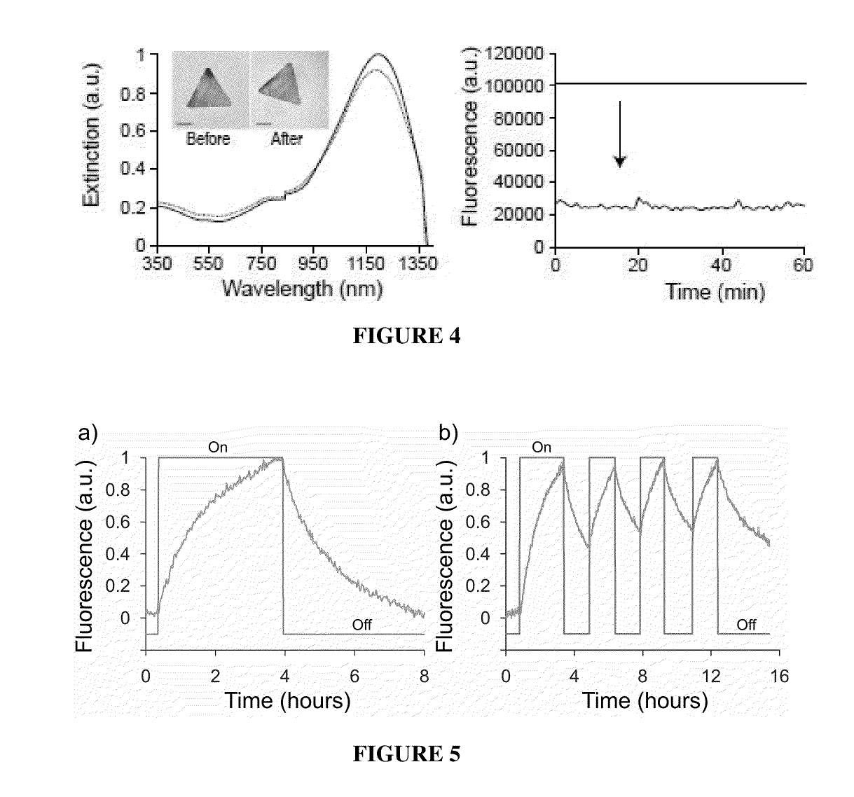

This local heating is dependent upon the resonance of the incident laser light with the SPR wavelength. Changes to the nanoparticle morphology will shift the SPR, and thus diminish the efficacy of the system. It is known that the high energy vertices of anisotropic noble metal nanostructures are susceptible to surface reorganization effects under mild conditions. Because elevated temperatures have been implicated in these processes, the morphological evolution of oligonucleotide-modified nanoprisms was monitored before and after laser irradiation by Transmission Electron Microscopy (TEM) and UV-vis-NIR spectroscopy. Interestingly, representative TEM analysis did not identify any discernable change in the nanoprism shape, edge length, or tip sharpness (FIG. 4A). Morphology was unchanged even after 9 hours of laser irradiation which is well beyond a typical release experiment (3-4 hours). Moreover, UV-vis-NIR spectra taken before and after irradiation indicate that the SPR dipole peak at 1200 nm does not change significantly (FIG. 4A). Because the nanoprism SPR dipole peak position is extremely sensitive to morphological features such as tip truncation, the lack of spectral peak shift demonstrates the structural stability of these conjugates when subjected to laser irradiation. The UV-vis-NIR spectra do indicate a slight drop in SPR peak intensity. This minimal drop in intensity, however, also occurs in a control experiment where the shutter on the laser head is closed and the sample does not receive any irradiation. Thus, this change cannot be attributed to laser induced morphology or concentration changes.

The morphological invariance of oligonucleotide-modified nanoprisms under laser irradiation is important in maintaining the function of these conjugates. Morphological reorganization in the case of gold nanoprisms has been determined to significantly shift the plasmon resonance by hundreds of nanometers towards higher energy wavelengths. Indeed, inducing nanoprism surface reorganization by removing excess ligands from solution blue-shifts the dipole plasmon resonance to around 700 nm. At 700 nm, the conjugates do not have any appreciable extinction at the laser wavelength. If nanoprism reorganization were to take place, the photothermal efficiency of the nanoprism would decrease as the SPR peak absorbance blue shifted away from the laser excitation (selected to excite the SPR of the starting nanoprisms) until ultimately the effect, e.g., excitation, became negligible. Because the plasmonic properties of the nanoprisms are unaffected, however, the nanostructures remain functional and the nanoprisms do not surface reorganize. Thus, the nanoprisms are not destroyed, mutated, or altered by the excitation of their SPRs during the methods disclosed herein.

To probe the importance of plasmonic wavelength in these experiments, analogous duplex-functionalized 13 nm spherical nanoparticles were prepared and studied in the context of a similar experiment. These particles have an SPR absorbance at 532 nm and no appreciable extinction at the laser wavelength (FIG. 3A, dashed line). In this case, laser irradiation does not yield a significant change in fluorescence, demonstrating that the effect requires a plasmonic nanostructure that absorbs at the laser irradiation wavelength (FIG. 3B, dashed line). The slight increase in fluorescence is attributed to heat generated by the nearby laser source. This contribution is diminutive when considering the change in fluorescence observed for oligonucleotide-modified nanoprisms. This experiment also accounts for any extrinsic heating of the solution, the cuvette, or the sample compartment that might otherwise explain the behavior observed in the case of the anisotropic gold nanoprism. Finally, this experiment confirms that in oligonucleotide-modified gold nanoparticles, light induced heating can discriminate between nanoparticle types based on their specific SPR.

It has been shown that femtosecond pulsed laser irradiation (λ=400 nm) is able to dissociate the Au—S bond for spherical gold nanoparticles functionalized with thiolated oligonucleotides. In order to rule out this process as the driving force behind the observed oligonucleotide release, the chemical stability of the Au—S bond on the nanoprism surface was investigated. Specifically, a cyanine 5.5 (Cy5.5) dye labeled thiolated oligonucleotide was synthesized to determine the stability of the covalently bound oligonucleotide during excitation using the previously described experimental set-up (FIG. 2). Under 1064 nm laser irradiation, no change in fluorescence intensity is observed which indicates that the thiolated oligonucleotide remains bound to the gold nanoprism surface (FIG. 4B, top signal).

The chemical stability of the Au—S bond under continuous wave 1064 nm laser irradiation illustrates an important finding for light controlled nucleic acid release. While oligonucleotide release is possible using a Au—S bond dissociation mechanism, there are several reasons why Au—S dissociation is undesirable. First, ligands are required to maintain stability towards particle aggregation. For the majority of applications under consideration, solution-phase stability both before and after irradiation are of crucial importance. Second, Au—S mediated release does not allow one to use the ability to denature an immobilized duplex that melts at a designed temperature for sequence tunable release. Finally, Au—S dissociation is an entropically irreversible process, and thus does not allow release and subsequent sequestration of a cargo.

The possibility of release followed by (re)sequestration of the agent was assessed by using fluorophore labeled oligonucleotides. These experiments also address the concern of thermal damage to oligonucleotides during release from the particle surface due to potentially high local temperatures. It was hypothesized that oligonucleotide damage would correlate strongly with an inability of the released nucleic acids to rehybridize back with the complementary oligonucleotides bound to the nanoprism. Several hours of nanoprism irradiation immediately followed by several hours of no irradiation resulted in a fluorescence signal which rises and falls back to a value similar to the original signal (FIG. 5A). This observation is attributed to the ability of the released oligonucleotides to rehybridize back to the nanoprisms after irradiation has ceased. Because the system is able to fully recover its fluorescence value, the released nucleic acids are not degraded by local heating effects. Furthermore, the response of the oligonucleotide-modified nanoprisms to 4 cycles of irradiation and darkness implies an extraordinary stability against changes in electrostatic surface charge and photothermally induced temperature gradients (FIG. 5B). The released oligonucleotides are therefore able to bind and participate in hybridization repeatedly, demonstrating significant functional persistence over time, and the ability to sequester a released nucleic acid in a reversible manner.

The disclosed nanostructures can be used in various applications. These applications include (a) light and SPR-mediated release of agents, such as oligonucleotides, in an in vitro or in vivo application for gene therapy, gene delivery, or gene regulation; (b) photothermal agents for induced cell death; (c) a platform for analyzing local temperature gradients produced by SPR mediated heating by tuning the heat labile association of attached molecules (such as modifying the percent complementarity of oligonucleotides and/or the number of nucleobases of complementary oligonucleotides); (d) light and SPR-mediated release of drugs or other therapeutic agents conjugated to the released molecules; (e) light and SPR-mediated catalysis of agent-conjugated molecules sequestered by nanoprisms; and (f) light and SPR-mediated mechanical actuation of associated agents.

### Nanostructures

Nanostructures disclosed herein have an agent, such as a diagnostic or therapeutic agent, associated (directly or indirectly) with a nanoprism. The agent can be, for example, one or more oligonucleotides, proteins, peptides, antibodies, or non-peptide drugs. In cases where the agent comprises an oligonucleotide, the oligonucleotide agent can be covalently bound to the nanoprism surface, either directly or through a linker or spacer moiety, or can be hybridized to a second oligonucleotide which is directly or indirectly bound to the nanoprism surface and having sufficient complementarity to hybridize the oligonucleotide agent.

The nanostructures disclosed herein can optionally include a label moiety on one of the components of the nanostructure (e.g., on the agent or associated with the nanoprism separately) that allows one to monitor the presence of the nanostructure. For example, an agent can be modified to include a label, such as a fluorophore, that does not produce a signal when near the nanoprism, but produces a signal once the fluorophore is no longer associated with the nanoprism. In a specific example, gold quenches fluorescence in a distance-related manner (e.g., the closer a fluorophore is to gold the stronger the gold's ability to quench the fluorophore's fluorescence). Thus, when a therapeutic or other agent having a fluorophore label is associated, either directly or indirectly, with a nanoprism in a nanostructure as disclosed herein, the fluorophore label does not produce a fluorescence signal. However, upon dissociation from the nanoprism, the fluorophore is no longer quenched by the nearby gold and a fluorescence signal can be detected.

Non-limiting examples of fluorophores include 5(6)-carboxyfluorescein, 2′,4′,1,4,-tetrachlorofluorescein; 2′,4′,5′,7′,1,4-hexachlorofluorescein, other fluorescein dyes (such as those disclosed in U.S. Pat. Nos. 5,188,934; 6,008,379; 6,020,481, incorporated herein by reference), 6-((7-amino-4-methylcoumarin-3-acetyl)amino)hexanoic acid (Cou), 5(and 6)-carboxy-X-rhodamine (Rox), other rhodamine dyes (such as those disclosed in U.S. Pat. Nos. 5,366,860; 5,847,162; 5,936,087; 6,051,719; 6,191,278; 6,248,884, incorporated herein by reference), benzophenoxazines (such as those disclosed in U.S. Pat. No. 6,140,500, incorporated herein by reference), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye, Cyanine 7 (Cy7) Dye, Cyanine 9 (Cy9) Dye, other cyanine dyes (such as disclosed in International Publication No. WO 97/45539), 6-carboxy-4′,5′-dichloro-2′,7′-dimethoxyfluorescein (JOE), 5(6)-carboxy-tetramethyl rhodamine (Tamara), or any one of the Alexa dye series, available from Molecular Probes, Eugene, Oreg.

Dissociation of an agent and/or labeled moiety from the nanoprism can occur under a variety of conditions. As discussed above, an agent and/or labeled moiety can be associated with a nanoprism through a non-covalent interaction, such as hydrogen bonding (for example, to a complementary molecule that is covalently attached to the nanoprism). In such cases, dissociation of the agent and/or labeled moiety from the nanoprism and/or nanostructure can occur upon disruption of the non-covalent interaction. Heat can be an effective way to disrupt non-covalent interactions, such as hydrogen-bonding. Heat can also be an effective way to break covalent interactions that involve heat-labile moieties.

### Nanoprisms

The nanoprisms of the disclosed nanostructures can be of any metal or metal combination that exhibits prismatic properties, including one or more surface plasmon resonance, such as gold and silver nanoprisms, and is anisotropic. Other contemplated metals include, but are not limited to, platinum, palladium, and copper.

Nanoprisms can be prepared via any known technique. For example, formation of triangular gold nanoprisms is disclosed in U.S. Pat. No. 7,588,624, which is incorporate by reference in its entirety. Silver nanoprism formation is described, for example, in U.S. Ser. No. 11/715,562, the disclosure of which is incorporated by reference in its entirety.

The nanoprisms can be any geometry, and in some cases are triangular, square, pentagonal, or hexagonal. Triangular gold nanoprisms can have a dipole surface plasmon in the near-infrared region, e.g., about 750 nm to about 1400 nm, or about 900 nm to about 1300 nm, or about 1000 nm to about 1250 nm. The surface plasmon resonance can be excited by irradiation with a narrow band of wavelengths of light. For example, as seen in FIG. 3A, gold triangular nanoprisms have a surface plasmon resonance around about 1000 nm to about 1350 nm.

Irradiation with one or more wavelengths within a surface plasmon resonance range can excite the surface plasmon resonance and generate localized temperature change at the nanoprism surface, as described above. As used herein, the term “narrow band of wavelengths” refers to light having about a 10 to 100 nm deviation of wavelengths, and also refers to light having a single wavelength. For example, a narrow band of wavelengths of about 1000 nm to about 1050 nm is light having wavelengths of about a 50 nm deviation. In another example, wavelengths of about 1050±50 nm provides a narrow band of wavelengths of about 1000 nm to about 1100 nm, with a 100 nm deviation. Narrow bands of wavelengths of light can be obtained using a wavelength filter in front of a light source. Additionally or alternatively, light can be produced as a single wavelength from a light source, such as a laser. The exact wavelength of light or narrow bands of wavelengths of light used in the disclosed methods are dependent upon the surface plasmon resonances of the nanoprisms used, and are easily determined by the person of skill.

Excitation of the SPR of nanoprisms disclosed herein provides an increase in the temperature surrounding the nanoprism. The temperature at or near the nanoprism can be, after irradiation, about 30° C. to about 500° C. The temperature can be about 30° C. to about 300° C., about 35° C. to about 200° C., about 35° C. to about 150° C., about 35° C. to about 140° C., about 35° C. to about 130° C., about 35° C. to about 120° C., about 40° C. to about 110° C., about 40° C. to about 100° C., about 40° C. to about 95° C., about 40° C. to about 90° C., about 40° C. to about 85° C., about 45° C. to about 80° C., about 45° C. to about 75° C., or about 45° C. to about 70° C.

Irradiation of the nanoprisms disclosed herein does not result in destruction or alteration of the nanoprisms. The SPR of the nanoprisms is substantially identical before and after the irradiation. As used herein, the term “substantially identical” refers to a deviation of the SPR of the nanoprism after irradiation of ±20%, ±15%, ±10%, or ±5% in comparison to the SPR of the nanoprism prior to irradiation.

For triangular nanoprisms, the edge length of the nanoprism can be about 30 nm to about 500 nm, or about 50 nm to about 400 nm, about 50 nm to about 350 nm, about 75 nm to about 300 nm, about 80 nm to about 250 nm, about 85 nm to about 225 nm, about 90 nm to about 200 nm, about 95 nm to about 175 nm, about 100 nm to about 150 nm. Specifically contemplated edge lengths include about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 300 nm, about 325 nm, about 350 nm, about 375 nm, about 400 nm, about 425 nm, about 450 nm, about 475 nm, and about 500 nm.

### Modified Nanoprisms

The nanoprisms can be modified with, for example, agents, e.g., therapeutic and/or diagnostic agents, such as oligonucleotides, proteins, antibodies, and the like, using known techniques. For example, modification of gold nanoprism surfaces by attachment of oligonucleotides, proteins, antibodies, and the like, is disclosed in, e.g., U.S. Pat. Nos. 6,361,944; 6,506,564; 6,767,702; and 6,750,016; and U.S. Patent Publication No. 2002/0172953; and in International Publication Nos. WO 98/04740; WO 01/00876; WO 01/51665; and WO 01/73123, the disclosures of which are incorporated by reference in their entirety.

These surface modified nanoprisms, then, can be used to deliver the agent to a cell and/or in detection of a target. In various embodiments, the target comprises at least two portions. The lengths of these portions and the distance(s), if any, between them are chosen so that when the surface-modified nanoprisms interact with the target a detectable change occurs. These lengths and distances can be determined empirically and will depend on the type of nanoprism used and its size and the type of electrolyte which will be present in solutions used in the assay. Also, when a target is an oligonucleotide and is to be detected in the presence of other oligonucleotides or non-target compounds, the portions of the target to which the oligonucleotide(s) on the oligonucleotide-modified nanoprism is to bind must be chosen so that they contain a sufficiently unique sequence such that detection of the nucleic acid will be specific. These techniques are well known in the art and can be found, for example, in U.S. Pat. Nos. 6,986,989; 6,984,491; 6,974,669; 6,969,761; 6,962,786; 6,903,207; 6,902,895; 6,878,814; 6,861,221; 6,828,432; 6,827,979; 6,818,753; 6,812,334; 6,777,186; 6,773,884; 6,767,702; 6,759,199; 6,750,016; 6,740,491; 6,730,269; 6,726,847; 6,720,411; 6,720,147; 6,709,825; 6,682,895; 6,677,122; 6,673,548; 6,645,721; 6,635,311; 6,610,491; 6,582,921; 6,506,564; 6,495,324; 6,417,340; and 6,361,944, each of which is herein incorporated by reference in its entirety.

### Oligonucleotides

As used herein, the term “oligonucleotide” refers to a single-stranded oligonucleotide having natural and/or unnatural nucleotides. Throughout this disclosure, nucleotides are alternatively referred to as nucleobases. The oligonucleotide can be a DNA oligonucleotide, an RNA oligonucleotide, or a modified form of either a DNA oligonucleotide or an RNA oligonucleotide.

Naturally occurring nucleobases include adenine (A), guanine (G), cytosine (C), thymine (T), and uracil (U), as well as non-naturally occurring nucleobases such as xanthine, diaminopurine, 8-oxo-N⁶-methyladenine, 7-deazaxanthine, 7-deazaguanine, N⁴,N⁴-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine, and the “unnatural” nucleobases include those described in U.S. Pat. No. 5,432,272 and Freier et al. Nucleic Acids Research, 25:4429-4443 (1997), each of which is incorporated by reference in its entirety. The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808; in Sanghvi, Antisense Research and Application, Crooke and B. Lebleu, eds., CRC Press, 1993, Chapter 15; in Englisch et al., Angewandte Chemie, International Edition, 30:613-722 (1991); and in the Concise Encyclopedia of Polymer Science and Engineering, J. I. Kroschwitz Ed., John Wiley & Sons, 1990, pages 858-859, Cook, Anti-Cancer Drug Design, 6, 585-607 (1991), each of which is hereby incorporated by reference in its entirety. Nucleobase also includes compounds such as heterocyclic compounds that can serve like nucleobases including certain “universal bases” that are not nucleosidic bases in the most classical sense but serve as nucleosidic bases. Especially mentioned as universal bases are 3-nitropyrrole, optionally substituted indoles (e.g., 5-nitroindole), and optionally substituted hypoxanthine. Other desirable universal bases include pyrrole, diazole, and triazole derivatives, including those universal bases known in the art. Modified forms of oligonucleotides are also contemplated which include those having at least one modified internucleotide linkage. In one embodiment, the oligonucleotide is all or in part a peptide nucleic acid. Other modified internucleoside linkages include at least one phosphorothioate linkage. Still other modified oligonucleotides include those comprising one or more universal bases. The oligonucleotide incorporated with the universal base analogues is able to function as a probe in hybridization, and as a primer in PCR and DNA sequencing. Examples of universal bases include but are not limited to 5′-nitroindole-2′-deoxyriboside, 3-nitropyrrole, inosine and pypoxanthine.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

Modified oligonucleotides includes oligonucleotides wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed oligonucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et al., Current Opinion in Structural Biology 5:343-355 (1995) and Susan M. Freier and Karl-Heinz Altmann, Nucleic Acids Research, 25:4429-4443 (1997), the disclosures of which are incorporated by reference in their entirety.

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002), the disclosures of which are incorporated by reference in their entirety.

The oligonucleotide can be bound to the nanoprism through a 5′ linkage and/or the oligonucleotide is bound to the nanoprism through a 3′ linkage. In various aspects, at least one oligonucleotide is bound through a spacer to the nanoprism. In these aspects, the spacer is an organic moiety, a polymer, a water-soluble polymer, a nucleic acid, a polypeptide, and/or an oligosaccharide. Methods of functionalizing the oligonucleotides to attach to a surface of a nanoprism are well known in the art. See Whitesides, Proceedings of the Robert A. Welch Foundation 39th Conference On Chemical Research Nanophase Chemistry, Houston, Tex., pages 109-121 (1995), or Mucic et al. Chem. Comm. 555-557 (1996) (describes a method of attaching 3′ thiol DNA to flat gold surfaces; this method can be used to attach oligonucleotides to nanoprisms), the disclosures of which are incorporated by reference in their entirety. The alkanethiol method can also be used to attach oligonucleotides to other metal, semiconductor and magnetic colloids and to the other nanoprisms listed above. Other functional groups for attaching oligonucleotides to solid surfaces include phosphorothioate groups (see, e.g., U.S. Pat. No. 5,472,881, incorporated by reference herein, for the binding of oligonucleotide-phosphorothioates to gold surfaces), substituted alkylsiloxanes (see, e.g. Burwell, Chemical Technology, 4:370-377 (1974) and Matteucci and Caruthers, J. Am. Chem. Soc., 103:3185-3191 (1981) for binding of oligonucleotides to silica and glass surfaces, and Grabar et al., Anal. Chem., 67:735-743 for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid surfaces. The following references describe other methods which may be employed to attached oligonucleotides to metal surfaces, such as nanoprisms: Nuzzo et al., J. Am. Chem. Soc., 109:2358 (1987) (disulfides on gold); Allara and Nuzzo, Langmuir, 1:45 (1985) (carboxylic acids on aluminum); Allara and Tompkins, J. Colloid Interface Sci., 49:410-421 (1974) (carboxylic acids on copper); Iler, The Chemistry Of Silica, Chapter 6, (Wiley 1979) (carboxylic acids on silica); Timmons and Zisman, J. Phys. Chem., 69:984-990 (1965) (carboxylic acids on platinum); Soriaga and Hubbard, J. Am. Chem. Soc., 104:3937 (1982) (aromatic ring compounds on platinum); Hubbard, Acc. Chem. Res., 13:177 (1980) (sulfolanes, sulfoxides and other functionalized solvents on platinum); Hickman et al., J. Am. Chem. Soc., 111:7271 (1989) (isonitriles on platinum); Maoz and Sagiv, Langmuir, 3:1045 (1987) (silanes on silica); Maoz and Sagiv, Langmuir, 3:1034 (1987) (silanes on silica); Wasserman et al., Langmuir, 5:1074 (1989) (silanes on silica); Eltekova and Eltekov, Langmuir, 3:951 (1987) (aromatic carboxylic acids, aldehydes, alcohols and methoxy groups on titanium dioxide and silica); Lec et al., J. Phys. Chem., 92:2597 (1988) (rigid phosphates on metals).

In some embodiments, the oligonucleotides are associated with the nanoprism disclosed herein by non-covalent interaction with a complementary oligonucleotide. In such cases, the associated oligonucleotide is not directly attached to the nanoprism, but rather is indirectly associated via hybridization to a complementary oligonucleotide that is directed attached to the nanoprism. Throughout this disclosure, description of various features of the oligonucleotides applies to oligonucleotides both directly attached and indirectly associated with the nanoprisms.

Nanoprisms disclosed herein can be functionalized with an oligonucleotide, or modified form thereof, which is from about 15 to about 100 nucleotides in length. Also contemplated are oligonucleotides of about 15 to about 90 nucleotides in length, about 15 to about 80 nucleotides in length, about 15 to about 70 nucleotides in length, about 15 to about 60 nucleotides in length, about 15 to about 50 nucleotides in length about 15 to about 45 nucleotides in length, about 15 to about 40 nucleotides in length, about 15 to about 35 nucleotides in length, about 15 to about 30 nucleotides in length, about 15 to about 25 nucleotides in length, about 15 to about 20 nucleotides in length, and all oligonucleotides intermediate in length of the sizes specifically disclosed to the extent that the oligonucleotide is able to achieve the desired result. Accordingly, oligonucleotides of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated.

### Oligonucleotide Features

The nanostructures disclosed herein can comprise an agent, such as an oligonucleotide, that can modulate expression of a gene product expressed from a target polynucleotide. Accordingly, antisense oligonucleotides which hybridize to a target polynucleotide and inhibit translation, siRNA oligonucleotides which hybridize to a target polynucleotide and initiate an RNAse activity (for example RNAse H), triple helix forming oligonucleotides which hybridize to double-stranded polynucleotides and inhibit transcription, and ribozymes which hybridize to a target polynucleotide and inhibit translation, are contemplated.

In various aspects, a plurality of oligonucleotides can be attached to the nanoprism. As a result, each oligonucleotide-modified nanoprism can have the ability to bind to a plurality of target compounds. In various aspects, the plurality of oligonucleotides can be identical. It is also contemplated wherein the plurality of oligonucleotides includes about 10 to about 100,000 oligonucleotides, about 10 to about 90,000 oligonucleotides, about 10 to about 80,000 oligonucleotides, about 10 to about 70,000 oligonucleotides, about 10 to about 60,000 oligonucleotides, 10 to about 50,000 oligonucleotides, 10 to about 40,000 oligonucleotides, about 10 to about 30,000 oligonucleotides, about 10 to about 20,000 oligonucleotides, about 10 to about 10,000 oligonucleotides, and all numbers of oligonucleotides intermediate to those specifically disclosed to the extent that the oligonucleotide-modified nanoprism is able to achieve the desired result.

Thus, each nanoprism provided herein can have a plurality of oligonucleotides attached to it. As a result, each nanoprism-oligonucleotide nanostructure has the ability to bind to a plurality of oligonucleotides and/or target polynucleotides having a sufficiently complementary sequence. For example, if a specific mRNA is targeted, a single nanoprism has the ability to bind to multiple copies of the same transcript. In one aspect, methods are provided wherein the nanoprism is functionalized with identical oligonucleotides, i.e., each oligonucleotide has the same length and the same sequence. In other aspects, the nanoprism is functionalized with two or more oligonucleotides which are not identical, i.e., at least one of the attached oligonucleotides differ from at least one other attached oligonucleotide in that it has a different length and/or a different sequence. In aspects wherein different oligonucleotides are associated with the nanoprism, these different oligonucleotides bind to the same single target polynucleotide but at different locations, or bind to different target polynucleotides which encode different gene products. Accordingly, in various aspects, a single functionalized nanoprism may be used in a method to inhibit expression of more than one gene product. Oligonucleotides are thus used to target specific polynucleotides, whether at one or more specific regions in the target polynucleotide, or over the entire length of the target polynucleotide as the need may be to effect a desired level of inhibition of gene expression.

Accordingly, the oligonucleotides are designed with knowledge of the target sequence. Methods of making oligonucleotides of a predetermined sequence are well-known. See, for example, Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are contemplated for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

Alternatively, oligonucleotides are selected from a library. Preparation of libraries of this type is well know in the art. See, for example, Oligonucleotide libraries: United States Patent Publication No. 2005/0214782, incorporated by reference herein. Preparation of siRNA oligonucleotide libraries is generally described in United States Patent Publication No. 2005/0197315, the disclosure of which is incorporated herein by reference in its entirety.

Further provided are embodiments wherein the oligonucleotide is functionalized to the nanoprism in such a way that the oligonucleotide is released from the nanoprism after the nanoprism enters a cell. In general, an oligonucleotides can be release from the surface of a nanoprism using either chemical methods, photon release (i.e., irradiating cells in which nanoprism have entered using an electromagnetic wavelengths chosen based on the nanoprism particle size), and changes in ionic or acid/base environment.

In one aspect of this embodiment, the oligonucleotide is attached to the nanoprism via an acid-labile moiety and once the functionalized nanoprism is taken into the cell via, for example, an endosome, acidification of the endosome (a normal part of endosomal uptake) releases the oligonucleotides. This aspect is particular useful in instances where the intent is to saturate the cell with for example, an siRNA. Release from the nanoprism would improve kinetics and resolve potential steric hindrance problems in embodiments where siRNA. RNAi for modulating gene expression is well known in the art and generally described in, for example, U.S. Patent Publication No. 2006/0019917, U.S. Patent Publication No. 2006/0008907 and U.S. Patent Publication No. 2005/0059016, the disclosures of which are incorporated herein by reference in their entireties.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of nanostructure disclosed herein. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of nanoparticle and a specific oligonucleotide.

### Oligonucleotide Sequences and Hybridization

Each oligonucleotide-modified nanoprism has the ability to hybridize to a portion of a second oligonucleotide having a sequence sufficiently complementary. In some cases, the second oligonucleotide is a target oligonucleotide (e.g., a portion of a polynucleotide that encode a gene product). In various aspects, the oligonucleotides of oligonucleotide-modified nanoprism are 100% complementary to a portion of the second oligonucleotide, i.e., a perfect match, while in other aspects, the oligonucleotides are at least (meaning greater than or equal to) about 95% complementary to portions of the second oligonucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to portions of the second oligonucleotide over the length of the oligonucleotide(s).

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

“Hybridization” means an interaction between two strands of nucleic acids by hydrogen bonds in accordance with the rules of Watson-Crick DNA complementarity, Hoogstein binding, or other sequence-specific binding known in the art. Hybridization can be performed under different stringency conditions known in the art. These hybridization conditions are well known in the art and can readily be optimized for the particular system employed. See, e.g., Sambrook et al., Molecular Cloning: A Laboratory Manual (2nd ed. 1989). Preferably stringent hybridization conditions are employed. Under appropriate stringency conditions, hybridization between the two complementary strands can reach about 60% or above, about 70% or above, about 80% or above, about 90% or above, about 95% or above, about 96% or above, about 97% or above, about 98% or above, or about 99% or above.

### Spacers

In certain aspects, functionalized nanoprisms are contemplated which include those wherein an agent, such as an oligonucleotide, peptide, protein, antibody, or non-peptide drug, is attached to the nanoprism through a spacer. “Spacer” as used herein means a moiety that does not have a therapeutic or diagnostic effect on a cell or participate in modulating gene expression per se but which serves to increase distance between the nanoprism and the agent (e.g., functional oligonucleotide), or to increase distance between individual agents when attached to the nanoprism in multiple copies. Thus, spacers are contemplated being located between individual agents (e.g., oligonucleotides) in tandem, whether the therapeutic agents are the same or different. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, or combinations thereof.

In certain aspects, the spacer has a moiety covalently bound to it, the moiety comprising a functional group which can bind to the nanoprisms. These are the same moieties and functional groups as described above. As a result of the binding of the spacer to the nanoprisms, the therapeutic agent is spaced away from the surface of the nanoprisms and is more accessible to the cell environment, e.g., an oligonucleotide more accessible for hybridization with its target. In instances wherein the spacer is a polynucleotide, the length of the spacer in various embodiments at least about 10 nucleotides, 10-30 nucleotides, or even greater than 30 nucleotides. The spacer may have any sequence which does not interfere with the ability of the oligonucleotides to become bound to the nanoprisms or to the target polynucleotide. The spacers should not have sequences complementary to each other or to that of the therapeutic agents oligonucleotides, but may be all or in part complementary to the target polynucleotide. In certain aspects, the bases of the polynucleotide spacer are all adenines, all thymines, all cytidines, all guanines, all uracils, or all some other modified base.

In another embodiment, a non-nucleotide linker of the invention comprises a basic nucleotide, polyether, polyamine, polyamide, peptide, carbohydrate, lipid, polyhydrocarbon, or other polymeric compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, the disclosures of which are all incorporated by reference herein.

A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine, for example at the C1 position of the sugar.

In various aspects, linkers contemplated include linear polymers (e.g., polyethylene glycol, polylysine, dextran, etc.), branched-chain polymers (see, for example, U.S. Pat. No. 4,289,872; U.S. Pat. No. 5,229,490; International Patent Publication No. WO 93/21259; lipids; cholesterol groups (such as a steroid); or carbohydrates or oligosaccharides. Other linkers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337, the disclosures of which are incorporated by reference herein. Other useful polymers as linkers known in the art include monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

In still other aspects, oligonucleotide such as poly-A or hydrophilic or amphiphilic polymers are contemplated as linkers, including, for example, amphiphiles (including oligonucletoides).

### Target Polynucleotides

In various aspects, the disclosed nanostructures are modified with an oligonucleotide that is a target for an intracellular polynucleotide or are co-administered with an oligonucleotide that is a target for an intracellular polynucleotide. The target polynucleotide is can be eukaryotic, prokaryotic, or viral.

In various embodiments, methods provided include those wherein the target polynucleotide is a mRNA encoding a gene product and translation of the gene product is inhibited, or the target polynucleotide is DNA in a gene encoding a gene product and transcription of the gene product is inhibited. In methods wherein the target polynucleotide is DNA, the polynucleotide is in certain aspects DNA which encodes the gene product being inhibited. In other methods, the DNA is complementary to a coding region for the gene product. In still other aspects, the DNA encodes a regulatory element necessary for expression of the gene product. “Regulatory elements” include, but are not limited to enhancers, promoters, silencers, polyadenylation signals, regulatory protein binding elements, regulatory introns, ribosome entry sites, and the like. In still another aspect, the target polynucleotide is a sequence which is required for endogenous replication.

The terms “start codon region” and “translation initiation codon region” refer to a portion of an mRNA or gene that encompasses contiguous nucleotides in either direction (i.e., 5′ or 3′) from a translation initiation codon. Similarly, the terms “stop codon region” and “translation termination codon region” refer to a portion of such an mRNA or gene that encompasses contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the oligonucleotides on the functionalized nanoparticles.

Other target regions include the 5′ untranslated region (5′UTR), the portion of an mRNA in the 5′ direction from the translation initiation codon, including nucleotides between the 5′ cap site and the translation initiation codon of an mRNA (or corresponding nucleotides on the gene), and the 3′ untranslated region (3′UTR), the portion of an mRNA in the 3′ direction from the translation termination codon, including nucleotides between the translation termination codon and 3′ end of an mRNA (or corresponding nucleotides on the gene). The 5′ cap site of an MRNA comprises an N7-methylated guanosine residue joined to the 5′-most residue of the MRNA via a 5′-5′ triphosphate linkage. The 5′ cap region of an mRNA is considered to include the 5′ cap structure itself as well as the first 50 nucleotides adjacent to the cap site.

For prokaryotic target polynucleotides, in various aspects, the polynucleotide is genomic DNA or RNA transcribed from genomic DNA. For eukaryotic target polynucleotides, the polynucleotide is an animal polynucleotide, a plant polynucleotide, a fungal polynucleotide, including yeast polynucleotides. As above, the target polynucleotide is either a genomic DNA or RNA transcribed from a genomic DNA sequence. In certain aspects, the target polynucleotide is a mitochondrial polynucleotide. For viral target polynucleotides, the polynucleotide is viral genomic RNA, viral genomic DNA, or RNA transcribed from viral genomic DNA.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of an oligonucleotide-functionalized nanoprism. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of nanoprism and a specific oligonucleotide.

### Non-Oligonucleotide Agents and Multiple Agents

The nanostructures can comprise more than one agent. The agent can be an oligonucleotide as described above or a protein, peptide, antibody, peptide mimetic, non-peptide drug, or combinations thereof. The agent can be covalently attached to the nanoprism, either directly or through a space or linker moiety, as described above. In cases where the agent is an oligonucleotide, the agent can be hybridized to first or second oligonucleotide of the nanostructure or attached to the nanoprism directly or through a spacer or linker moiety. In cases where more than one agent is associated with the nanostructure, one agent can be a therapeutic agent and another agent can be a diagnostic agent, two or more agents can be therapeutic agents, and/or two or more agents can be diagnostic agents.

The agent can be selected based on its binding specificity to a ligand expressed in or on a target cell type or a target organ. Moieties of this type include a receptor for a ligand on a target cell (instead of the ligand itself), and in still other aspects, both a receptor and its ligand are contemplated in those instances wherein a target cell expresses both the receptor and the ligand. In other aspects, members from this group are selected based on their biological activity, including for example enzymatic activity, agonist properties, antagonist properties, multimerization capacity (including homo-multimers and hetero-multimers). With regard to proteins, agents contemplated include full length protein and fragments thereof which retain the desired property of the full length proteins. Fusion proteins, including fusion proteins wherein one fusion component is a fragment or a mimetic, are also contemplated. This group also includes antibodies along with fragments and derivatives thereof, including but not limited to Fab′ fragments, F(ab)₂ fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

Non-peptide drugs are compounds that provide a therapeutic benefit, but are not peptides (e.g., are not repeating units of amino acids). Non-peptide drugs can include some peptide-like features, such as, for example, vancomycin, which contains some peptide bonds.

All patents, publications and references cited herein are hereby fully incorporated by reference. In case of conflict between the present disclosure and incorporated patents, publications and references, the present disclosure should control.

### EXAMPLES

### Preparation of Oligonucleotide-Modified Au Nanoprism Conjugates

Synthesis and functionalization of Au nanoprism conjugates followed literature methods.(21, 22) Briefly, nanoprisms were synthesized using the seed mediated growth method, and were allowed to separate from spherical nanoprisms that form concomitantly by settling in a conical centrifuge tube overnight. After purification of the Au nanoprisms, thiolated oligonucleotides (SEQ ID NO: 1: 5′-TTA TGA CAT TTC CTA A₁₀ SH-3′, Cy5.5 labeled Sequence; SEQ ID NO: 2: 5′-TTA TGA CAT TTC CTA A₁₀-Cy5.5-SH-3′) were treated with dithiothreitol (100 mM) in disulfide cleaving buffer (170 mM phosphate buffer, pH 8.0) for 1 hour and purified with a desalting column. Approximately 1.2 OD₂₆₀ (3.93 μM) of the purified thiolated oligonucleotide and 1.2 OD₂₆₀ (6.55 μM) of the fluorophore-labeled complementary oligonucleotide (SEQ ID NO: 3: 5′-6-FAM TAG GAA ATG TCA TAA-3′) were added to Au nanoprisms that had been centrifuged (3 min at 8000 RPM) twice to remove excess cetyltrimethylammonium bromide. After allowing this mixture to react for 1 hour, the solution was slowly brought to 150 mM NaCl and 10 mM phosphates in the presence of surfactant (0.01% sodium dodecyl sulfate) over the course of several hours.(16, 17) After allowing functionalization to occur overnight, oligonucleotide-modified Au nanoprism solutions were purified of excess oligonucleotides by centrifugation three times (3 min at 8000 RPM) and resuspension in physiological salt buffer (150 mM NaCl, 10 mM phosphates, and 0.01% sodium dodecyl sulfate). Oligonucleotide-modified nanoprisms were then diluted to 0.5 OD₁₂₀₀, which corresponds to a concentration of 45 pM, and aliquoted for future experiments. All oligonucleotides were synthesized in-house with phosphoramidites purchased from Glen Research Corp. and all buffer reagents were purchased from Sigma-Aldrich Inc.

### Simultaneous SPR Excitation and Measurement of Fluorescence Intensity

A 500 mW 1064 nm continuous wave diode pumped solid state Nd:YVO₄ laser (Newport Corp., Excelsior Scientific class) was coupled to a fluorescence spectrometer (Horiba Jobin Yvon Inc., Fluorolog-3). A pyrex dielectric mirror (Newport Corp., wavelength range: 1030-1090 nm) was used to reflect and align the beam through the sample chamber. Measurement of the laser power using a power detector (Newport Corp., 818P series) confirmed a high reflectivity from the minor with a loss of at most 5% of the laser power.

In all fluorescence experiments, samples were allowed several hours inside the instrument to reach thermal equilibrium so as to ensure that changes in fluorescence were due to photothermally generated heat.

### Method for Determining the Percent of Released Oligonucleotides

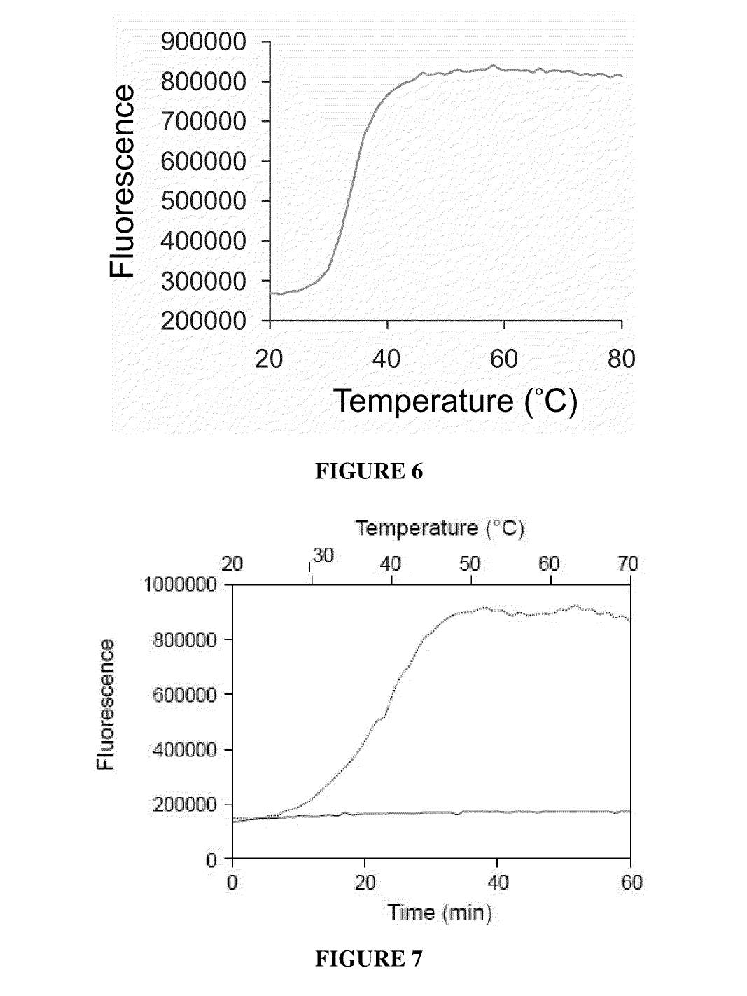

Using an associated temperature bath, the fluorescence intensity from oligonucleotide-modified Au nanoprism aliquots was monitored as a function of temperature (FIG. 6). The characteristic “S” shaped curve indicates that the oligonucleotide duplexes attached to the nanoprism surface are dehybridizing at a specific temperature, as expected. In order to approximate the average fraction of the total oligonucleotides released from the Au nanoprism, we assumed that the lowest value on the curve represents the equilibrium at which the maximum number of fluorophore-labeled complementary oligonucleotides have hybridized to the thiolated oligonucleotides on the particle surface. Conversely, we assumed the highest value on the curve to represent the equilibrium at which the maximum number of fluorophore-labeled complementary oligonucleotides have dehybridized from the nanoprisms and are present in solution. Using this maximum possible change in fluorescence as a basis of comparison, laser induced dehybridization curves could be analyzed in a similar manner and quantified for percent of total oligonucleotides released. For every new set of oligonucleotide-modified Au nanoprism samples prepared and aliquoted, one was removed and analyzed in this way so that only samples within a common set were compared to one another. It is important to note that this method provides an average value for the entire solution and does not give information about how individual particles respond to the laser irradiation.

### Investigation of Free Oligonucleotide Duplexes

In order to further elucidate the role of the inorganic gold nanostructure in SPR mediated nucleic acid dehybridization, oligonucleotide duplexes were prepared in the absence of nanoparticles. One oligonucleotide labeled with fluorescein was synthesized to be complementary to a second oligonucleotide labeled with the fluorescence quencher dabcyl such that when the duplex formed between them, fluorescein and dabcyl would be in close proximity (SEQ ID NO: 4: 5′-6-FAM ATC GAT CCT AGAT-3′, SEQ ID NO: 5: 5′-TAG CTA GGA TCTA-Dabcyl-3′). These sequences were added together in 150 mM NaCl, 10 mM phosphates, and 0.01% sodium dodecyl sulfate and allowed to hybridize overnight. A sample of approximately 7 nM of duplex irradiated for 60 min by the 500 mW 1064 nm laser showed no change in fluorescence indicating the stability of the duplexed oligonucleotides to laser irradiation (FIG. 7). Immediately following this experiment, the fluorescence of the sample was monitored as a function of temperature to confirm that the fluorophore and quencher labeled oligonucleotides had hybridized as designed (FIG. 7). The “S” shaped curve observed under these conditions confirmed the oligonucleotide duplexes dehybridize under increased temperatures, but not under 1064 nm laser irradiation.

### REFERENCES

[1] I. H. El-Sayed, X. Huang, M. A. El-Sayed, Cancer Lett. 2006, 239, 129.
[2] X. Huang, I. H. El-Sayed, W. Qian, M. A. El-Sayed, J. Am. Chem. Soc. 2006, 128, 2115.
[3] L. R. Hirsch, R. J. Stafford, J. A. Bankson, S. R. Sershen, B. Rivera, R. E. Price, J. D. Hazle, N. J. Halas, J. L. West, Proc. Natl. Acad. Sci. U.S.A. 2003, 100, 13549.
[4] P. K. Jain, W. Qian, M. A. El-Sayed, J. Am. Chem. Soc. 2006, 128, 2426.
[5] G. Wu, A. Mikhailovsky, H. A. Khant, C. Fu, W. Chiu, J. A. Zasadzinski, J. Am. Chem. Soc. 2008, 130, 8175.
[6] X. Yang, S. E. Skrabalak, Z.-Y. Li, Y. Xia, L. V. Wang, Nano Lett. 2007, 7, 3798.
[7] L. Au, D. Zheng, F. Zhou, Z.-Y. Li, X. Li, Y. Xia, ACS Nano 2008, 2, 1645.
[8] S. Link, M. A. El-Sayed, Int. Rev. Phys. Chem. 2000, 19, 409.
[9] J. Stehr, C. Hrelescu, R. A. Sperling, G. Raschke, M. Wunderlich, A. Nichtl, D. Heindl, K. Kurzinger, W. J. Parak, T. A. Klar, J. Feldmann, Nano Lett. 2008, 8, 619.
[10] A. Wijaya, S. B. Schaffer, I. G. Pallares, K. Hamad-Schifferli, ACS Nano 2008, ASAP.
[11] S. E. Lee, G. L. Liu, F. Kim, L. P. Lee, Nano Lett. 2008, ASAP.
[12] S. R. Sershen, S. L. Westcott, N. J. Halas, J. L. West, J. Biomed. Mater. Res. 2000, 51, 293.
[13] A. M. Derfus, G. von Maltzahn, T. J. Harris, T. Duza, K. S. Vecchio, E. Ruoslahti, S. N. Bhatia, Adv. Mater. 2007, 19, 3932.
[14] K. Hamad-Schifferli, J. J. Schwartz, A. T. Santos, S. Zhang, J. M. Jacobson, Nature 2002, 415, 152.
[15] V. S. Kalambur, E. K. Longmire, J. C. Bischof, Langmuir 2007, 23, 12329.
[16] C. A. Mirkin, R. L. Letsinger, R. C. Mucic, J. J. Storhoff, Nature 1996, 382, 607.
[17] N. L. Rosi, D. A. Giljohann, C. S. Thaxton, A. K. R. Lytton-Jean, M. S. Han, C. A. Mirkin, Science 2006, 312, 1027.
[18] D. S. Seferos, D. A. Giljohann, H. D. Hill, A. E. Prigodich, C. A. Mirkin, J. Am. Chem. Soc. 2007, 129, 15477.
[19] N. L. Rosi, C. A. Mirkin, Chem. Rev. 2005, 105, 1547.
[20] A. Wijaya, K. Hamad-Schifferli, Langmuir 2008, 24, 9966.
[21] J. E. Millstone, D. G. Georganopoulou, X. Xu, W. Wei, S. Li, C. A. Mirkin, Small 2008, 4, 2176.
[22] J. E. Millstone, W. Wei, M. R. Jones, H. Yoo, C. A. Mirkin, Nano Lett. 2008, 8, 2526.
[23] J. E. Millstone, S. Park, K. L. Shuford, L. Qin, G. C. Schatz, C. A. Mirkin, J. Am. Chem. Soc. 2005, 127, 5312.
[24] B. Dubertret, M. Calame, A. J. Libchaber, Nat. Biotechnol. 2001, 19, 365.
[25] D. S. Seferos, D. A. Giljohann, N. L. Rosi, C. A. Mirkin, ChemBioChem 2007, 8, 1230.
[26] W. Wei, S. Li, L. Qin, C. Xue, J. E. Millstone, X. Xu, G. C. Schatz, C. A. Mirkin, Nano Lett. 2008, 8, 3446.
[27] X. Peng, J. Wickham, A. P. Alivisatos, J. Am. Chem. Soc. 1998, 120, 5343.
[28] S. Chen, D. L. Carroll, Nano Lett. 2002, 2, 1003.
[29] K. L. Shuford, M. A. Ratner, G. C. Schatz, J. Chem. Phys. 2005, 123, 114713.
[30] K. L. Kelly, E. Coronado, L. L. Zhao, G. C. Schatz, J. Phys. Chem. B 2003, 107, 668.

1. A method comprising
irradiating a nanostructure comprising (a) a nanoprism and (b) an agent with light having a narrow band of wavelengths or a single wavelength that excites a surface plasmon resonance of the nanoprism, wherein
prior to the irradiating, the agent is associated with the nanoprism, and after the irradiating, the agent is dissociated from the nanoprism.

2. The method of claim 1, wherein the agent comprises a diagnostic agent or a therapeutic agent.

3. The method of claim 2, wherein the agent comprises an oligonucleotide, a protein, a peptide, or mixtures thereof.

4. (canceled)

5. The method of claim 2, wherein the therapeutic agent comprises an oligonucleotide, a protein, a peptide, a non-peptide drug, or mixtures thereof.

6. The method of claim 5, wherein the therapeutic agent comprises a protein, a peptide, a non-peptide drug, or mixtures thereof attached to a spacer.

7. (canceled)

8. (canceled)

9. (canceled)

10. The method of claim 1, wherein the agent comprises a first oligonucleotide having a first sequence,
the nanostructure comprises a second oligonucleotide having a second sequence and attached to at least a portion of the nanoprism surface;
all or a portion of the second sequence is sufficiently complementary to the first sequence to allow hybridization of the first oligonucleotide and the second oligonucleotide; and
after the irradiating, the first oligonucleotide dehybridizes from the second oligonucleotide.

11. (canceled)

12. (canceled)

13. The method of claim 10, wherein the first oligonucleotide is complementary to a polynucleotide encoding a gene product.

14. The method of claim 13, wherein the first oligonucleotide is sufficiently complementary to inhibit expression of the gene product.

15. (canceled)

16. (canceled)

17. (canceled)

18. (canceled)

19. The method of claim 10, wherein the first oligonucleotide is greater than 95% complementary to the polynucleotide, greater than 90% complementary to the polynucleotide, greater than 80% complementary to the polynucleotide, greater than 75% complementary to the polynucleotide, greater than 70% complementary to the polynucleotide, greater than 65% complementary to the polynucleotide, greater than 60% complementary to the polynucleotide, greater than 55% complementary to the polynucleotide, or greater than 50% complementary to the polynucleotide.

20. (canceled)

21. (canceled)

22. (canceled)

23. (canceled)

24. The method of claim 1, wherein the nanostructure comprises two or more agents.

25. The method of claim 24, wherein at least one of the agents is a therapeutic agent.

26. (canceled)

27. The method of claim 1, wherein the nanostructure further comprises a fluorophore.

28. (canceled)

29. (canceled)

30. The method of claim 27, further comprising monitoring a change in fluorescence of the fluorophore and correlating the change in fluorescence to release or sequestration of the agent from or to the nanoprism, wherein an increase in fluorescence corresponds to a release of the agent and a decrease in fluorescence corresponds to a sequestration of the agent.

31. The method of claim 1, wherein the irradiating produces a temperature surrounding the nanoprism of about 40° C. to about 85° C.

32. The method of claim 1, wherein the surface plasmon resonance of the nanoprism after irradiating is substantially identical to the surface plasmon resonance of the nanoprism prior to irradiating.

33. The method of claim 1, wherein the nanoprism is triangular.

34. The method of claim 1, wherein the nanoprism comprises gold or silver.

35. The method of claim 34, wherein the nanoprism comprises gold.

36. The method of claim 33, wherein the nanoprism has an edge length of about 90 nm to about 200 nm.

37. (canceled)


SEQUENCE LISTING (5 sequences)

SEQ ID NO: 1 - DNA, 24 bp, Artificial Sequence
  Synthetic oligonucleotide
ttatgacatttcctaaaaaaaaaa

SEQ ID NO: 2 - DNA, 24 bp, Artificial Sequence
  Synthetic oligonucleotide
ttatgacatttcctaaaaaaaaaa

SEQ ID NO: 3 - DNA, 15 bp, Artificial Sequence
  Synthetic oligonucleotide
taggaaatgtcataa

SEQ ID NO: 4 - DNA, 13 bp, Artificial Sequence
  Synthetic oligonucleotide
atcgatcctagat

SEQ ID NO: 5 - DNA, 13 bp, Artificial Sequence
  Synthetic oligonucleotide
tagctaggatcta